(19) United States
(12) Patent Application Publication
(10) Pub. No.: US 2022/0181032 A1
(43) Pub. Date: Jun. 9, 2022

(54) INGESTING AND PROCESSING CLINICAL INFORMATION BY A CLINICAL ENGINE

(71) Applicant: AMO Development, LLC, Santa Ana, CA (US)

(72) Inventors: Joshua Young, Great Neck, NY (US); Charles Scales, Ponte Vedra, FL (US)

(21) Appl. No.: 17/457,403

(22) Filed: Dec. 2, 2021

Related U.S. Application Data
(60) Provisional application No. 63/121,492, filed on Dec. 4, 2020.

(51) Int. Cl. G16H 50/30; G16H 10/60

(52) U.S. Cl. CPC G16H 50/30; G16H 50/20; G16H 10/60

(57) ABSTRACT

A system is provided herein. The system includes a memory that stores processor executable instructions for a clinical engine and a processor coupled to the memory. The processor executes the clinical engine to cause the system to register patient profiles. Each of the patient profiles includes a unique identification. The processor executes the clinical engine to cause the system to aggregate data instances from devices that performed procedures to produce clinical information. Each of the data instances are associated with a code that includes the unique identification. The processor also executes the clinical engine to cause the system to process the clinical information to determine and associate health or procedure risks with patient types.

### CROSS REFERENCE TO RELATED APPLICATIONS

This application claims priority from U.S. Provisional Patent Application No. 63/121,492, entitled “INGESTING AND PROCESSING CLINICAL INFORMATION BY AN ACCUMULATION ENGINE,” filed on Dec. 4, 2020, which is hereby incorporated by reference as if set forth in full in this application for all purposes.

### FIELD OF INVENTION

The disclosure is related to systems and methods for implementing machine learning and/or artificial intelligence (ML/AI). More particularly, the systems and methods that implement ML/AI relate to ingesting and processing clinical information by a clinical engine.

### BACKGROUND

Medical information is a robust field that includes paper and electronic records for patients, medical/service providers, insurance companies, procedures, surgeries, etc. Conventionally, a burden falls on a patient to accumulate medical information as the patient seeks care from different medical/service providers. Each of the different medical/service providers, in turn, must perform data entry and processing activities (e.g., transcription) to intake and distribute the medical information, which is time consuming and prone to human errors. Further, compatibility can be a major problem when medical information is exchanged between the different medical/service providers, as well as during a billing process to the insurance companies. Unfortunately, there are presently no comprehensive techniques that provide complete, accurate, concise, timed, and dated medical information across the noted robust field, while maintaining a high level of quality and accuracy.

### SUMMARY

According to an embodiment, a system is provided. The system includes a memory configured to store processor executable instructions for a clinical engine; and at least one processor coupled to the memory. The at least one processor is configured to execute the clinical engine to cause the system to register one or more patient profiles, each of the one or more patient profiles comprising a unique identification; aggregate one or more data instances from one or more devices that performed one or more procedures, each of the one or more data instances being associated with a code that includes the unique identification, to produce clinical information; and process the clinical information to determine and associate health or procedure risks with one or more patient types.

According to one or more embodiments, the system embodiment above can be implemented as an apparatus, a method, and/or a computer program product.

### BRIEF DESCRIPTION OF THE DRAWINGS

A more detailed understanding may be had from the following description, given by way of example in conjunction with the accompanying drawings, wherein like reference numerals in the figures indicate like elements, and wherein:

FIG. 1 illustrates a diagram of a system according to one or more embodiments;

FIG. 2 illustrates a diagram of a system of performing ingesting operations according to one or more embodiments;

FIG. 3 illustrates a diagram of a system of performing processing operations according to one or more embodiments;

FIG. 4 illustrates a diagram of a method according to one or more embodiments;

FIG. 5 illustrates a diagram of a method according to one or more embodiments;

FIG. 6 illustrates a diagram of a method according to one or more embodiments; and

FIG. 7 illustrates a diagram of a method according to one or more embodiments.

### DETAILED DESCRIPTION

Disclosed herein is a ML/AI method and system. More particularly, disclosed herein are systems including and methods implementing ML/AI algorithms (e.g., a clinical engine) that ingest and process clinical information. In this regard, the clinical engine is processor executable code or software that is necessarily rooted in process operations by, and in processing hardware of, medical device equipment to provide centralized clinical information storage with distributed processing.

According to one or more embodiments, the clinical engine is a gravity point of a clinical architecture or ecosystem and third party services and/or devices. In turn, the clinical engine generates, receives, and communicates clinical information across the clinical architecture and the third party services and/or devices.

Generally, clinical information can be any information or data predictive of pathology that the clinical engine uses. Examples of clinical information include non-identifying demographic information and raw patient data in varying mediums/forms, such as electronic health records and mobile patient-journey applications, and refined diagnostic information based on the raw patient data, such as health or procedure risks. According to one or more embodiments, clinical information includes one or more data instances (e.g., raw data) relative to one or more procedures for a patient, where each data instance is associated with a unique identification.

The technical effects and benefits of the centralized clinical information storage and distributed processing of the clinical engine include providing complete, accurate, concise, timed, and dated clinical information across the clinical architecture or ecosystem and third party services and/or devices. Further, the clinical engine is data agnostic, as the clinical engine can incorporate any data format by any device to provide clinical information that would otherwise not be available for risk assessments. Additionally, the technical effects and benefits of the clinical engine include eliminating data entry burdens and transcription error risks.

As an example, on a day of a cataract surgery, a femtosecond laser treatment is performed. Following femtosecond laser treatment, a write-enabled display shows a matrix barcode with biometric and treatment data gathered during the femtosecond laser treatment. A medical professional scans the displayed matrix bar code with a clinical engine client on a mobile device. The clinical engine client ingests the biometric and treatment data and employs this biometric and treatment data to refine an intraocular lens calculation, which results in an enhanced intraocular lens calculation. The enhanced intraocular lens calculation is then displayed by the mobile device. The clinical engine client frees the medical professional of the transcription burden of the biometric and treatment data and avoids transcription errors. The biometric and treatment data and the enhanced intraocular lens calculation are communicated by the clinical engine client to a clinical engine server for ML/AI evaluation.

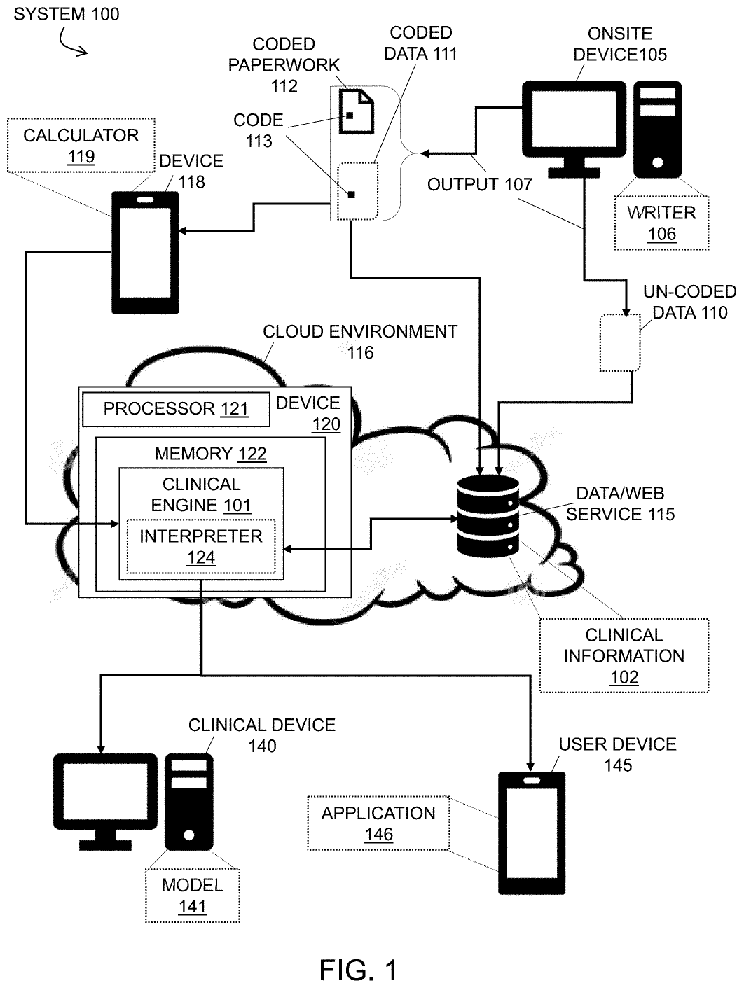

FIG. 1 is a diagram of a system 100 including a clinical engine 101 according to one or more embodiments. The system 100 can generally be viewed as a collection of disparate diagnostic, therapeutic, and surgical medical device equipment (e.g., surgical microscope heads-up display and individual-level provider heads up displays of the clinical architecture and the third party services/devices) connected and in communication with the clinical engine 101. The clinical engine 101 can generally be viewed as a clinical engine server for ML/AI evaluation. All or parts of the system 100 may be used to automatically generate, ingest, interpret, and distribute clinical information 102 (e.g., whether coded or un-coded).

As shown, the system 100 includes an onsite device 105 executing a writer 106 (e.g., client computer instructions/software of the clinical engine 101) that outputs (see arrows 107) the clinical information 102 in the forms of un-coded data 110, coded data 111, and coded paperwork 112. Note the coded data 111 and the coded paperwork 112 include a code 113. The clinical information 102 from the writer 106 can be communicated, as coded data 11, to a data/web service 115 of a cloud environment 116 for storage. The code 113, which can detail the clinical information 102 as well as a unique identification, can be scanned or received by a device 118 executing a calculator 119 (e.g., client computer instructions/software of the clinical engine 101). The clinical information 102 from the calculator 119 can be directly communicated to the device 120 executing the clinical engine 101. The device 120 comprises at least one processor 121 and one memory 122. The clinical engine 101 can further include an interpreter 124 (e.g., sub-software of the clinical engine 101). The clinical engine 101 can be in communication (as shown by arrow 129) with the data/web service 115. The clinical engine 101 can be in communication with a clinical device 140 executing a model 141 and a user device 145 executing an application 146. Note that, while single elements of the system 100 are shown, these elements represent one or more of that element. Each element of the system 100 is now further described.

As discussed herein, the clinical information 102 can be any information or data predictive of pathology and/or of value to performing one or more procedures that the clinical engine uses. For instance, the clinical information 102 can be aggregated for procedural needs in the absence of pathology, and vice versa. Examples of the clinical information 102 include non-identifying demographic information and raw patient data in varying mediums/forms, such as electronic health records and mobile patient-journey applications. Further, the clinical information 102 can include administrative data, claims data, patient/disease registries, health surveys, clinical trial data, etc. collected during the course of ongoing patient care. Examples of the clinical information 102 include diagnostic and/or clinical data for at least vision care, as well as refined diagnostic information based on any raw patient data that indicate and/or predict health or procedure risks. Further, the clinical information 102 can include clinical parameters obtained from disparate diagnostic, therapeutic, and surgical devices gathered, as well as from sources such as the electronic health records or non-writer-enabled machines.

Clinical information 102 examples respective to diagnostic and/or clinical data for vision care includes, but are not limited to, eye dimension information and other physical characteristics of the eye (mapping out multiple indexes of the eye or mapping), ocular characteristics or anatomy, prescription information, eye disease information, eye disease symptoms, cataract information, glaucoma information (e.g., intraocular pressure), dry eye information, surgery system data, and the like. Clinical information 102 examples respective to diagnostic and/or clinical data for vision care can also include information regarding custom intraocular lenses, custom contact lenses, custom corneal implants, and the like, which can be configured to treat or ameliorate any of a variety of vision conditions in a particular patient based on their unique ocular characteristics or anatomy.

Clinical information 102 examples respective to eye dimension information and/or ocular characteristics or anatomy include, but are not limited to, ocular biometry information, anterior corneal surface information, posterior corneal surface information, anterior lens surface information, posterior lens surface information, lens tilt information, and lens position information.

Clinical information 102 examples respective to surgery system data include, but are not limited to, alternative eye treatment procedure data, spectacle lens information, intraocular lens information, contact lens information, corneal ring implant information, collagenous corneal tissue thermal remodeling information, corneal inlay/onlay information, and corneal implant or graft information, along with parameters related to dioptic power, refractive index, anterior and posterior radius, lens thickness, asphericity, toricity, echelette design, haptic angulation, and lens filter. Further, examples of surgery system data include, but are not limited to various degrees of intraoperative rotation/tip/tilt associated with implantation of an intraocular lens and/or a variety of optical treatment modalities, along with vision treatment shapes or designs that can be administered to a patient.

Thus, the clinical information 102 contemplates a variety of vision related data, treatments, diagnostic data, and measurements that can be used by the system 100 to determine a vision state and used to correct vision.

Generally, the clinical engine 101 is a gravity point of the system 100. In this way, the clinical engine 101 implements centralized and distributed processing for ingesting the clinical information 102 from the devices 105 and 118 throughout the system 100. The clinical engine 101 can be software, hardware, or any combination thereof that utilizes ML/AI algorithms to automatically code, ingest, interpret, and distribute the clinical information 102). According to an embodiment, an entirety of the clinical engine 101 can be executed on a single device (e.g., the device 120) of the system 100, such that all the features of the clinical engine 101, the writer 106, the calculator 119, the model 141, and the application 146 are implemented therein. In an example operation, the clinical engine 101 can register a patient profile and aggregate the clinical information 102 relative to a patient profile. Registration can occur at any level of the clinical engine 101 and, in some cases, outside of the clinical architecture (i.e., outside of the system 100). Aggregation can be generally implemented by the clinical engine 101 after one or more procedures are completed and can be specifically implemented by the writer 106 and the calculation 119 in real-time during one or more procedures.

According to one or more embodiments, the clinical engine 101 can include specific software instances that implement particular operations of the clinical engine 101 itself. For instance, the clinical engine 101 can be a clinical engine server that operates as central accumulator software and communicates with clinical engine clients, such as the writer 106, the calculator 119, the model 141, and the application 146. Each of these clinical engine clients or client software instances can employ one or more of artificial intelligence, modeling, machine learning algorithms, and clinical calculation algorithms that mirror capabilities of the clinical engine server or the central accumulator software, while offloading processing responsibility. Thus, the system 100 and the clinical engine 101 work in conjunction with the writer 106, the calculator 119, the model 141, and the application 146 to provide a multi-stage software solution. Any results derived by the clinical engine 101, the model 141, and the application 146 can be displayed on the clinical device 140 and/or the user device 145.

The writer 106 includes computer instructions for producing/generating the clinical information 102 with respect to one or more procedures. Generally, the one or more procedures are representative of one or more of medical, routine, and surgical procedures. The writer 106 can aggregate and package (e.g., ingesting operations) the clinical information 102 relative to these procedures and associate a code 113 thereto. In this regard, the system shows the coded paperwork 112 and the coded data 111 as an example of the clinical information 102.

The calculator 119 includes computer instructions for processing/analyzing the clinical information 102 generated by the writer 102 (e.g., the coded paperwork 112 and the coded data 111). In this regard, the calculator 119 can include computer instructions, such as a clinical calculation algorithm for device-level calculations and unpacking of the clinical information associated with the code 113. For example, the calculator 119 can download from the clinical engine 101 the clinical information 102 derived from other devices to perform local device-level calculations to facilitate calculation of clinical parameters obtained from disparate diagnostic, therapeutic, and surgical devices, as well as the clinical information 102 gathered from sources (e.g., electronic health records or even non-writer-enabled machines with outputs that may be semi- or fully automatically ingested by the clinical engine 101).

The coded paperwork 112 and the coded data 111 can be communicated/received by at least the device 118 executing the calculator 119 (e.g., client computer instructions/software of the clinical engine 101).

The code 113 can be any visual representation of data that is machine-readable, such as a quick response code, a barcode, a two-dimensional pattern, a matrix barcode, etc. According to one or more embodiments, the code 113 can be a unique code that includes at least a unique identification for a patient.

According to one or more embodiments, the writer 106 generates the code 113 (e.g., a matrix barcode) on the fly from data (e.g., chemical screening information) resident on the onsite device 105 (e.g., medical diagnostic equipment as described herein) in which the writer 106 is running. In this way, the code 113, as it is generated on the fly, can include all the clinical information 102 aggregated and packaged by the writer 106, such that an improved and different data management and user experience is provided over conventional bar codes. Further, the code 113 can be scanned, for example, by a camera of the device 118, which causes the device 118 to receive/process the clinical information 102 within the code 113 (and/or the coded paperwork 112). For example, the calculator 119 can ingest the coded paperwork 112 and the coded data 111 to acquire the clinical information 102, the calculator 119 can apply a clinical calculation algorithm for device-level calculations, and the calculator 119 can securely upload the clinical information 102 (which includes the calculations) to the clinical engine 101. In this way, a technical effect and benefit of the system 100 includes offloading processing capabilities to the device 118 from the cloud environment 116. Additionally, the calculator 119 can download from the clinical engine 101 the clinical information 102 derived from other devices (e.g., the onsite device 105) to perform local device-level calculations to facilitate calculations. Thus, the calculator 119 being software resident on the device 118 reads the code 113 (e.g., the matrix barcode) and either locally processes/ingests the clinical information 102 (e.g., chemical screening information) therein or directly uploads the clinical information 102 for remote processing.

The model 141 (e.g., a machine learning model and/or resulting clinical model of the clinical engine 101) can be built on the clinical information 102. Building the model 141 can include physical hardware or software modeling, algorithmic modeling, and/or the like that seeks to represent the clinical information 102 (or subsets thereof) that has been collected and trained. In some aspects, building of the model 101 is part of self-training operations by the clinical engine 101.

The application 146 that acts as a client software instance of the clinical engine 101. Client software instances can employ one or more of artificial intelligence, modeling, machine learning algorithms, and clinical calculation algorithms that mirror capabilities of the clinical engine 101, while offloading processing responsibility.

According to one or more embodiments, the onsite device 105, the data/web service 115, the device 118, the device 120, the clinical device 140, and the user device 145 can structurally be any computing device comprising software and/or hardware, such as a general-purpose computer, with suitable interface circuits for transmitting and receiving signals to and from other items of the system 100. The hardware of these devices can include at least one processor and at least one memory, both of which are represented by way of example by the processor 121 and the memory 122 within the device 120. For example, the onsite device 105 can be any computing device comprising software and/or hardware, with suitable interface circuits for transmitting and receiving signals to and from other items of the system 100. The onsite device 105 can include a processor and a memory, with respect to executing the writer 106. Similar to the onsite device 105, the device 115 can be any computing device comprising software and/or hardware, such as a processor and a memory use to execute the calculator 119.

The processor 121 is representative of any computing circuit, central processing unit, microprocessor, and/or the like. The memory 122 is any non-transitory tangible media, such as magnetic, optical, or electronic memory (e.g., any suitable volatile and/or non-volatile memory, such as random-access memory or a hard disk drive). The memory 122 stores the computer instructions (e.g., of the clinical engine 101 and the interpreter 124) for execution by the processor 121, as well as clinical data 102 as needed. The processor 121, in executing the clinical engine 101, can be configured to receive, process, and manage the clinical information 102. The processor 121, in executing the clinical engine 101, can communicate the clinical information 102 to the memory 122 for storage and/or across the cloud environment 116.

Examples of the onsite device 105 and clinical device 140 may be, for example, a mobile phone, a smart phone, smartwatch, tablet or other portable smart device, as well as medical diagnostic equipment. Medical diagnostic equipment include, but are not limited to optical coherence tomography (OCT) devices, laser interferometers, corneal topography devices, phacoemulsification machines, corneal tomography devices, each of which and be utilized pre-, intra-, and post-operatively. By way of example and representation, the onsite device 105 and/or the clinical device 140 can be a CATALYS™ Precision Laser System by Johnson & Johnson Surgical Vision, Inc.

Examples of the device 118 and user device 145 may be, for example, a stationary or standalone computer processor, a desktop or laptop computer, medical diagnostic equipment (e.g., an auto analyzer machine), surgical tools, etc. including modem and/or router capabilities. By way of example and representation, the device 118 can be a mobile phone with a camera and a display. By way of example and representation, the user device 145 can be a hospital workstation.

By way of example and representation, the device 120 can be any combination of software and/or hardware that executes the clinical engine 101 to individually or collectively code, ingest, interpret, and distribute the clinical information 102. The device 120 can store and execute the clinical engine 101, as a clinical engine server. The interface circuits of the device 120 include input/output (I/O) communication interfaces that enables receiving signals from and/or transferring signals to other devices, such as by utilizing any number and combination of networks and various communication technologies, as described herein. The device 120 can be easily scalable, extensible, and modular, with the ability to change to different services or reconfigure some features independently of others.

By way of example and representation, the data/web service 115 can be any database or computing system that stores an organized collection of structured and/or unstructured information (i.e., the clinical information 102), For instance, the data/web service 115 can be a cloud-based clinical data repository of the clinical information 102 that supports centralized and distributed processing of the device 120 and the clinical engine 101.

According to one or more embodiments, the clinical information 102 can be organized by the data/web service 115 and/or the clinical engine 101 with respect to patient profiles, such that the clinical information 102 relative to one or more procedures for a patient are associated with the code 113 that includes a unique identification of a profile of that patient. The unique identification includes non-identifying demographic information, so that each patient profile acts as a specific repository for any accumulated clinical information 102 with non-identifying demographic information for a specific patient. In turn, a patient profile with the unique identification is generated for a specific patient. The unique identification identifies/indicates that specific patient. The patient profile receives all the clinical information 102 produced by one or more procedures for the specific patient. In cases where the clinical information 102 is generated by the onsite device 105, the clinical engine 101 (e.g., the writer 106) can cause any resulting data and/or paperwork to be coded with the code 113 that references the unique identification. In cases where a third party device produces un-coded data and/or paperwork, the clinical engine 101 can receive and interpret the un-coded data 110 and/or the paperwork with respect to the patient profile to further aggregate the clinical information 102.

Further, in operation, the clinical engine 101 (e.g., the central accumulator software) processes the patient data to determine health or procedure risks for the specific patient and associates these health or procedure risks with the patient profile. In some cases, the clinical engine 101 provides/distributes the health or procedure risks to a clinical device or a user device (e.g., executing model or application software thereon).

The cloud environment 116 may be a wired network, a wireless network, and/or include one or more wired and wireless networks, such as an intranet, a local area network (LAN), a wide area network (WAN), a metropolitan area network (MAN), a short-range network, a direct connection or series of connections, a cellular telephone network, or any other network or medium capable of facilitating communication between the items of FIG. 1 using any one of various communication standards/protocols (e.g., Bluetooth, Wi-Fi, Zigbee, Z-Wave, near field communications (NFC), Zigbee, infrared (IR), Ethernet, Universal Serial Bus (USB), or any other communication standards/protocols). Additionally, several networks may work alone or in communication with each other to facilitate communication in the cloud environment 116. In some instances, the device 120 and/or the data/web service 115 may be implemented as a single physical server on the cloud environment 116. In other instances, the device 120 and/or the data/web service 115 may be implemented as a virtual server a public cloud computing provider (e.g., Amazon Web Services (AWS)®) of the cloud environment 116.

According to one or more embodiments, the system 100 illustrates a number of communication examples.

As a first example, when the onsite device 105 and the device 118 are within the clinical architecture or ecosystem, these devices can directly communicate the coded paperwork 112 and the coded data 111 to the clinical engine 101. Further, the onsite device 105 and the device 118 can also directly communicate the coded paperwork 112 and the coded data 111 to the data/web service 115. The clinical engine 101 and the data/web service 115 communicate as needed.

As a second example, the onsite device 105 can be a third party service or device. A third party service/device is any service or device that is not within the clinical architecture or ecosystem (e.g., a computing device that does not subscribe to the protocols, standards, and the like of the system 100). In turn, the onsite device 105 generates the un-coded data 110. The un-coded data 110 is clinical information with no associated unique code (e.g., absent the code 113). The un-coded data 110 can still be communicated to the data/web service 115. In some cases, the data/web service 115 can be used as a repository before the un-coded data 110 is interpreted by the interpreter 124. That is, the interpreter 124 of the clinical engine 101 can process this clinical information and, if necessary, associated the code 113. In an example, the interpreter 123 processes the un-coded data 110 to extract clinical information and remove patient identifying information.

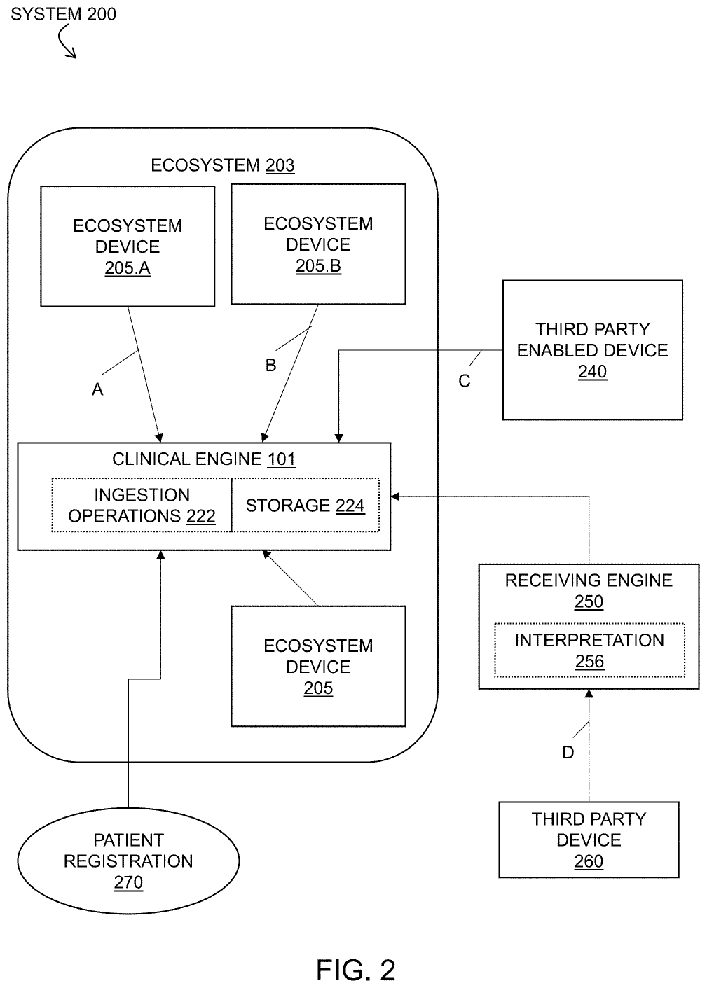

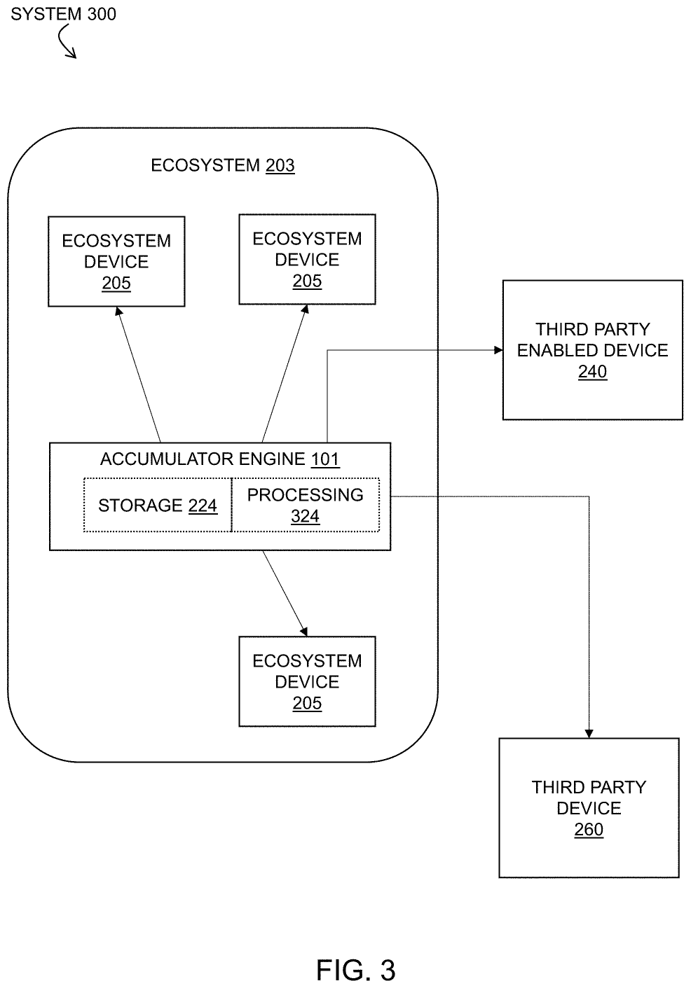

Turning now to FIGS. 2-3, a system 200 of performing ingestion operations and a system 300 performing processing operations are shown according to one or more embodiments. Note that, for ease of explanation and brevity, items of the system 100 that are similar to those of systems 200 and 300 are reused and not reintroduced.

The system 200 of FIG. 2 includes an ecosystem 203 comprising one or more ecosystem devices 205 that communicate with the clinical engine 101 that provides ingestion operations 222 and storage 224 operations therein. The clinical engine 101 is also in communication with one or more third party enabled devices 240 that are outside of the ecosystem 203, but capable of communicating with the ecosystem 203. In some cases, a receiving engine 250 is configured to perform interpretation operations 256 for one or more third party devices 260. With respect to any ingestion operations 222 or storage 224 of the clinical information 102, the system 200 can implement a patient registration 270 to create a patient profile. The patient profile can then be associated with received and/or stored clinical information 102.

The system 300 of FIG. 3 further includes processing 324 operations by the clinical engine 101 of the ecosystem 203. The processing 324 includes analyzing the clinical information 102 for patterns, errors, anomalies, etc. to determine health or procedure risks.

In an example operation of the system 200 of FIG. 2, the one or more ecosystem devices 205 directly provide (see arrow A) raw clinical information to the clinical engine 101. For instance, the ecosystem device 205.A (e.g., with the writer 106 thereon) can be deployed in an examination room to gather data during a procedure for a particular patient. As data is gathered (and in some cases displayed by the ecosystem device 205), the ecosystem device 205 can provide this data, as raw clinical information, to the clinical engine 101. The clinical engine 101 then stores 224 the raw clinical information with respect to a patient profile of the patient (as generated during patient registration 270). The clinical engine 101 can also perform an ingestion operations 222 of the raw clinical information. Ingestion operations 222 includes analyzing the raw clinical information (e.g., the data gathered during the procedure in the examination room) for patterns, errors, anomalies, etc. to determine health or procedure risks with the patient profile, which further produces the clinical information 102.

In another example operation of the system 200 of FIG. 2, the one or more ecosystem devices 205 directly provide (see arrow B) ingested clinical data to the clinical engine 101. For instance, the ecosystem device 205.B (e.g., with the writer 106 thereon) can be deployed in an examination room to gather and ingest data during a procedure to determine health or procedure risks with the patient profile. As data is gathered and ingested, the ecosystem device 205 can provide this ingested clinical data the clinical engine 101 and display this ingested data. The clinical engine 101 then stores 224 the ingested clinical data as the clinical information 102.

In another example operation of the system 200 of FIG. 2, third party enabled devices 240 and 260 provide the clinical information 102 to the clinical engine 101 in any form. For instance, the third party enabled device 240 (e.g., with writer software thereon) can be deployed in an examination room to gather data during a procedure for a particular patient. As this data is gathered, the third party enabled device 240 can provide (see arrow C) this data to the clinical engine 101, as the writer 106 thereon can configure the data for ingestion by the clinical engine 101. Additionally, the third party device 260 can provide (see arrow D) raw clinical information to the clinical engine 101 through the receiving engine 250. In this regard, when the third party device 260 (e.g., without the writer 106 thereon) gathers and provides the raw clinical information, the receiving engine 250 performs an interpretation operation 256 on the raw clinical information to prepare the data for ingestion operations 222 by the clinical engine 101.

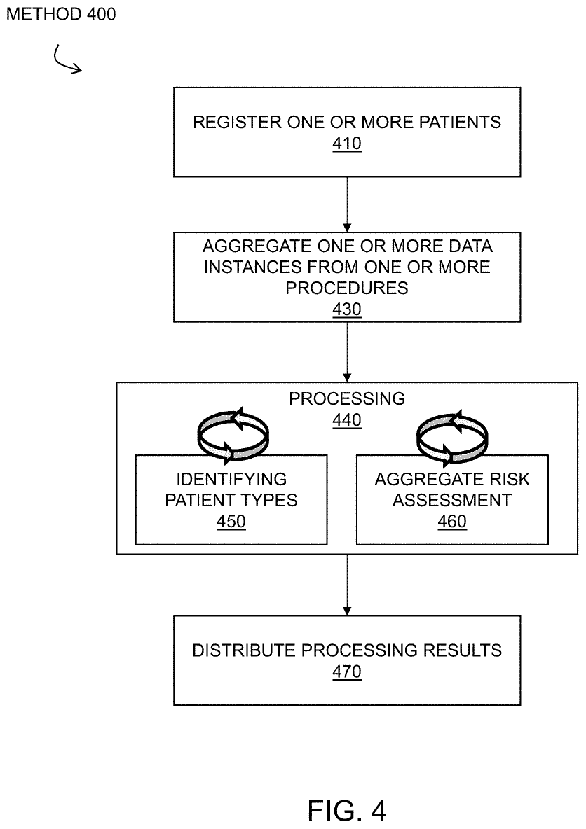

In an example operation of the system 300 of FIG. 3, the clinical engine 101 processes 324 the clinical information 102 received from the ecosystem devices 205, the third party enabled devices 240, the receiving engine 250, and the third party devices 260 to determine health or procedure risks. The clinical engine 101 then provides the health or procedure risks to the ecosystem devices 205, the third party enabled devices 240, the receiving engine 250, and the third party devices 260. Operations of the clinical engine 101 are further described herein, for example, with respect to FIGS. 4-7.

FIG. 4 illustrates a diagram of a method 400 according to one or more embodiments. The method 400 is an example of an operation of the clinical engine 101 described herein. For ease of explanation, reference is made to the items of FIGS. 1-3.

The method 400 begins at block 410, where the clinical engine 101 performs a registration of a patient. In some cases, one or more patients are registered in one or more profiles. Each patient registration may include creating a patient profile with and/or associating a unique identification (e.g., non-identifying demographic information).

The unique identification can be generated by any computer process, such as random generator that produces a sequence of numbers, letters, and/or symbols that cannot be reasonably predicted but for a random chance. A log or list of all unique identification can be kept by the clinical engine 101 so that duplicates are not produced, such as in the data/web service 115. In some cases, a date and time can be utilized in generating/finalizing the unique identification to guarantee a uniqueness. Patient registration can occur at any software instance (e.g., the writer 106, the calculator 119, the clinical engine 101, the model 141, and the application 146) of the clinical engine 101, and the patient profile can be stored similarly with any software instance.

At block 430, the clinical engine 101 aggregates the clinical information 102. In this regard, the clinical engine 101 aggregates one or more data instances from one or more devices (e.g., the onsite device 105) that performed one or more procedures to produce the clinical information 102. Further, the writer 106 and the calculator 119 can communicate the one or more data instances, and the clinical engine 101 can pull the one or more data instances from the data/web service 115. According to one or more embodiments, each of the one or more data instances have a code 113 that includes a unique identification. In this way, corresponding patient profiles based on these codes 113 can be found and matched.

According to one or more embodiments, the clinical engine 101 aggregates one or more data instances into the clinical information 102 to determine specific patient data. The specific patient data can be an aggregation of specific patient clinical information from the one or more procedures.

At block 440, the clinical engine 101 processes the clinical information 102. The processing of block 440 can further include identifying patient types at block 450 and/or aggregating risk assessments at block 460. Health and/or procedure risks include, but are not limited to, demographic factors (e.g., ethnicity, socioeconomic status), medication use, lifestyle habits (e.g., smoking, exercise, etc.), comorbidities, and other descriptors that might contribute to risk assessment. Patient types include, but are not limited to, preoperative patients, postoperative patients, intraoperative patients, patients with a known pathology, patients undergoing screening, patients undergoing emergency evaluation, and patients undergoing routine examination, as well as a category of person based on age, gender, health, etc. and/or a category of eye based on type of use, age, condition, etc.

According to one or more embodiments, specific patient data of the clinical information 102 is used to identify the one or more patient types and is processed to discover health and/or procedure risks with one or more patient types.

At block 470, according to one or more embodiments, the clinical engine 101 provides/distributes results of the processing at block 440. In this regard, the health and/or procedure risks and the patient types (e.g., all or portions of the clinical information 102) can be provided to the clinical device 140 or the user device 145 (e.g., to the model 141 or the application 146) so that a medical professional can review the results. In some cases, the results can be used to assess a patient and procedure in real-time. For instance, the clinical engine 101 presents different procedural risks for cataracts procedures for preoperative patient types who are under forty (40) vs. over sixty (60). Further, for post cataracts procedures (i.e., when those same patients become postoperative patient types), the clinical engine 101 presents different procedural risks for postoperative treatments. The medical professional can be any person or persons providing medical treatment and/or care. Example of the medical professional include, but are not limited to surgeons, doctors, medical clinicians, medical technicians, medical staff, nurses, and medical assistants.

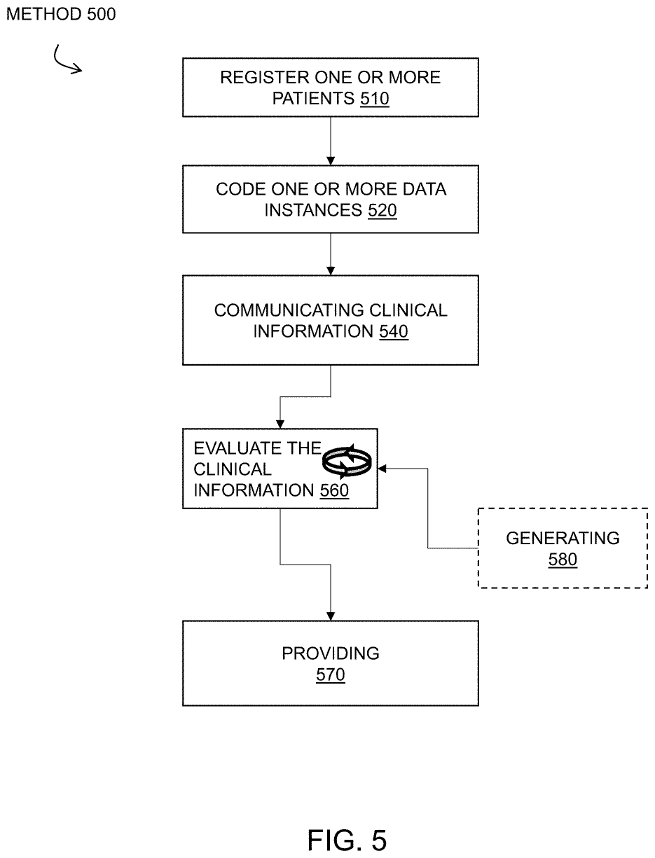

FIG. 5 illustrates a diagram of a method 500 according to one or more embodiments. The method 500 is an example of an operation of the clinical engine 101 described herein. For ease of explanation, reference is made to the items of FIGS. 1-4.

The method 500 begins at block 510, the clinical engine 101 performs a registration of a patient. Patient registration can occur at any software instance (e.g., the writer 106, the calculator 119, the clinical engine 101, the model 141, and the application 146 of the clinical engine 101) and includes creating a patient profile. Patient registration enables the writer 106, the calculator 119, and the clinical engine 101 to generate specific patient data with respect to the patient profile and within the clinical information.

At block 520, the clinical engine 101 (e.g., the writer 106) performs a coding of one or more data instances. Coding includes generating and/or associating a code 113 to the one or more data instances. The code 113 can be based on the unique identification of a patient profile. The code 113 can be a key that separates patient identifying information (e.g., according to the Health Insurance Portability and Accountability Act (HIPAA)) from information relative to the procedure, such that the information relative to the procedure can be easily transmitted without violating HIPAA regulations. In cases where the one or more data instances are generated at the onsite device 106, the writer 106 can cause any resulting data and/or paperwork to be coded with the code 113, thereby producing coded data 111 and paperwork 112. Note that the code 113 is scannable by the device 118 with the calculator 119 thereon.

By way of example and according to one or more embodiments, a patient receives lab results of a metabolic panel blood test in a printout. When the printout is accompanied by a matrix barcode (e.g., the code 113), the printout can be considered the coded paperwork 111. The code 113 itself can contain the lab results and the unique identification for that patient.

At block 540, the clinical engine 101 communicates the clinical information 102. For example, the calculator 119 transmits to the clinical engine 101 the clinical information 102 (e.g., the lab results) within the code 113 (after scanning). The clinical information 102 can then reside in folders/databases/storage with respect to the patient profile based one the unique identification for that patient. The clinical information 102 can reside, for instance, on the data/web service 115.

At block 560, the clinical engine 101 evaluates the communicated clinical information 102. In accordance with one or more embodiments, the clinical engine 101 implements ML/AI to ingest and process the clinical information 102, such as by processing of eye dimension information. For example, the clinical engine 101 utilizes ocular biometry information, anterior corneal surface information, posterior corneal surface information, anterior lens surface information, and/or posterior lens surface information of the clinical information 102 to determine intraocular lens power for a cataract procedure.

In general, the ML/AI algorithms therein can include neural networks. A neural network is a network or circuit of neurons, or in a modern sense, an artificial neural network (ANN), composed of artificial neurons or nodes or cells.

For example, an ANN involves a network of processing elements (artificial neurons) which can exhibit complex global behavior, determined by the connections between the processing elements and element parameters. These connections of the network or circuit of neurons are modeled as weights. A positive weight reflects an excitatory connection, while negative values reflects inhibitory connections. Inputs are modified by a weight and summed using a linear combination. An activation function may control the amplitude of the output. For example, an acceptable range of output is usually between 0 and 1, or it could be −1 and 1. In most cases, the ANN is an adaptive system that changes its structure based on external or internal information that flows through the network.

In more practical terms, neural networks are non-linear statistical data modeling or decision-making tools that can be used to model complex relationships between inputs and outputs or to find patterns in data. Thus, ANNs may be used for predictive modeling and adaptive control applications, while being trained via a dataset. Note that self-learning resulting from experience can occur within ANNs, which can derive conclusions from a complex and seemingly unrelated set of information. The utility of artificial neural network models lies in the fact that they can be used to infer a function from observations and also to use it. Unsupervised neural networks can also be used to learn representations of the input that capture the salient characteristics of the input distribution, and more recently, deep learning algorithms, which can implicitly learn the distribution function of the observed data Learning in neural networks is particularly useful in applications where the complexity of the data (e.g., the clinical information 102) or task (e.g., monitoring, diagnosing, and treating any number of various diseases) makes the design of such functions by hand impractical.

Neural networks can be used in different fields. Thus, the ML/AI algorithms therein can include neural networks that are divided generally according to tasks to which they are applied. These divisions tend to fall within the following categories: regression analysis (e.g., function approximation) including time series prediction and modeling; classification including pattern and sequence recognition; novelty detection and sequential decision making; data processing including filtering; clustering; blind signal separation, and compression. For example, Application areas of ANNs include nonlinear system identification and control (vehicle control, process control), game-playing and decision making (backgammon, chess, racing), pattern recognition (radar systems, face identification, object recognition), sequence recognition (gesture, speech, handwritten text recognition), medical diagnosis and treatment, financial applications, data mining (or knowledge discovery in databases, “KDD”), visualization and e-mail spam filtering. For example, it is possible to create a semantic patient profile from the clinical information 102.

According to one or more embodiments, the neural network can implement a long short-term memory neural network architecture, a convolutional neural network (CNN) architecture, or other the like. The neural network can be configurable with respect to a number of layers, a number of connections (e.g., encoder/decoder connections), a regularization technique (e.g., dropout); and an optimization feature.

The long short-term memory neural network architecture includes feedback connections and can process single data points (e.g., such as images), along with entire sequences of data (e.g., such as speech or video). A unit of the long short-term memory neural network architecture can be composed of a cell, an input gate, an output gate, and a forget gate, where the cell remembers values over arbitrary time intervals and the gates regulate a flow of information into and out of the cell.

The CNN architecture is a shared-weight architecture with translation invariance characteristics where each neuron in one layer is connected to all neurons in the next layer. The regularization technique of the CNN architecture can take advantage of the hierarchical pattern in data and assemble more complex patterns using smaller and simpler patterns. If the neural network implements the CNN architecture, other configurable aspects of the architecture can include a number of filters at each stage, kernel size, a number of kernels per layer.

At block 570, the clinical engine 101 provides/distributes health or procedure risks (e.g., all or portions of the clinical information). With reference to FIG. 1, the device 118 distributes the health or procedure risks to the clinical device 140 and/or the user device 145. In this regard, when the patient visits a primary care doctor office, the patient dons a portable heads-up display (e.g., the user device 145) and instantly has access to the lab results and any ML/AI based analysis of the clinical information 102 (from block 560) that alerts the patient to the health or procedure risks.

At optional block 580 (as designated by the dashed line), additional clinical information can be generated. For instance, when a patient returns to a medical office or seeks additional procedures, additional clinical information can be supplied to the clinical engine 101. As an example, when the patient undergoes medical imaging for neck pain subsequent to the metabolic panel blood test of block 520, the medical imaging report can be accompanied by a matrix barcode (e.g., the code 113). Yet, in this example, the matrix barcode does not contain all of the clinical information from the medical imaging report. Rather, the matrix barcode points the clinical engine 101 to a secure destination (e.g., a website or the data/web service 115) from which the all of the clinical information 102 can be obtained. Then, when the patient visits the primary care doctor office, the patient dons the portable heads-up display (e.g., the user device 145) and instantly has access to the medical imaging report, the lab results, and any ML/AI based analysis of the clinical information 102 that alerts the patient to the health or procedure risks.

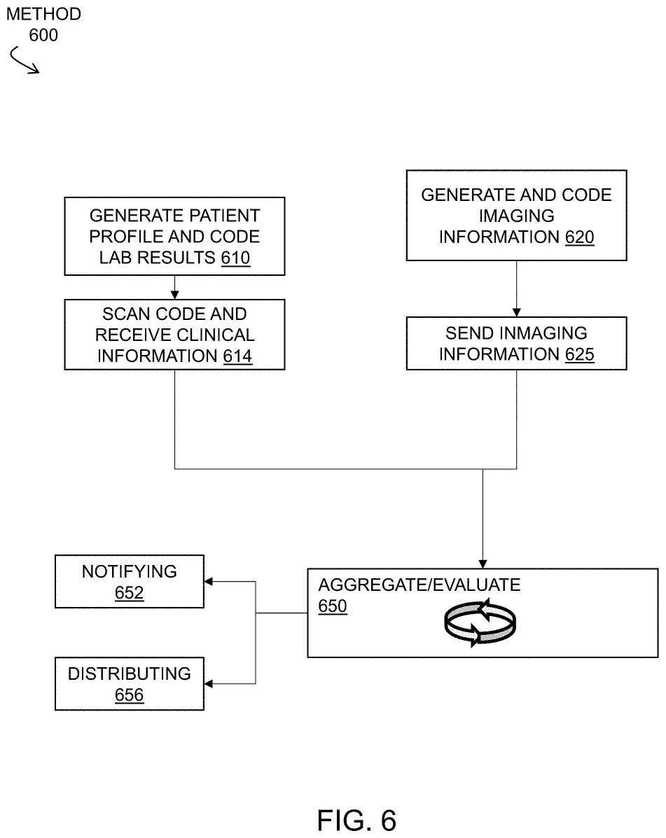

FIG. 6 illustrates a diagram of a method 600 according to one or more embodiments. The method 600 is an example of an operation of the clinical engine 101 described herein. For ease of explanation, reference is made to the items of FIGS. 1-5.

Further, reference is also made to a deployment of the clinical engine 101 with respect to providing clinical benefits for intraocular lens power calculations at a time of cataract surgery. Note that intraocular lens calculation formulae are currently under development that would require a medical professional to enter the clinical information 102 into a computer or mobile device at the time of cataract surgery. Such data may be generated by a laser, a phacoemulsification machine, or other diagnostic, therapeutic, or surgical instruments (e.g., the onsite device 105 enabled with the writer 106 to display any relevant clinical information on displays connected thereto).

The method 600 begins at block 610, where the clinical engine 101 performs generating and coding operations. In this case, a patient profile is generated with lab results of a metabolic panel blood test. That is, the writer 106 of the onsite device 105 (e.g., an auto analyzer machine) generates the coded paperwork 111 with a first code 113 thereon, on the fly, before the cataract surgery (e.g., some time before surgery, the patient undergoes pre-operative biometry at an ophthalmologist's office).

At block 614, the clinical engine 101 receives the clinical information 102 (e.g., the lab results). By way of example, for instance, the coded paperwork 111 is presented to a mobile phone (e.g., the device 118 executing the calculator 119) of a medical professional at the ophthalmologist's office, which scans the first code 113. Scanning the code first 113 causes the clinical information 102 to be communicated to the clinical engine 101. For example, the calculator 119 of the mobile phone transmits the lab results to the clinical engine 101.

At block 620, parallel generating and coding operations are performed. As an example, when the patient undergoes medical imaging with respect to the pre-operative biometry. Any resulting imaging information can be electronically coded with a second code 113. At block 625, the imaging information with the second code 113 can be automatically sent to a secure destination within the data/web service 115. Also, a receipt can be accompanied by the second code 113 that, when scanned, points the clinical engine 101 (e.g., the calculator 119) to the secure destination from which all of the medical imaging information can be obtained. Note that the secure destination can also be a website outside of the cloud environment 116.

At block 650, the clinical engine 101 accumulates/aggregates/obtains the clinical information 102. As noted herein, in cases where a third party device produces un-coded data and/or paperwork, the clinical engine 101 can receive and interpret the un-coded data and/or paperwork with respect to one or more patient profiles. For instance, while some biometry is performed on writer-enabled devices, keratometry and axial length are measured on a laser interferometer without the capabilities of the writer 106. An output of this interferometer is scanned or otherwise sent to the clinical engine 101.

Further, the clinical engine 101 performs an evaluation. The evaluation can include applications of ML/AI by the clinical engine 101. According to one or more embodiments, the evaluation can include, but is not limited to, analyzing the clinical information 102 by artificial intelligence, natural language processing, optical character recognition, etc. to extract the relevant clinical data. For example, having gathered extant clinical information 101 throughout the system 100, the clinical engine 101 (or other software instance therein) performs an initial intraocular lens calculation, e.g., utilized ML/AI.

At block 652, the clinical engine 101 provides notifications. Notifications can include any electronic transmission of information, such as by email, pop-up, text message, or the like. In an example, the clinical engine 101 provides an email to a medical professional regarding health risk identified by the evaluation of block 650. At block 656, the clinical engine 101 distribute the clinical information 102 to all software instances, so that these instances can further identify and accumulate data with respect to one or more patient types.

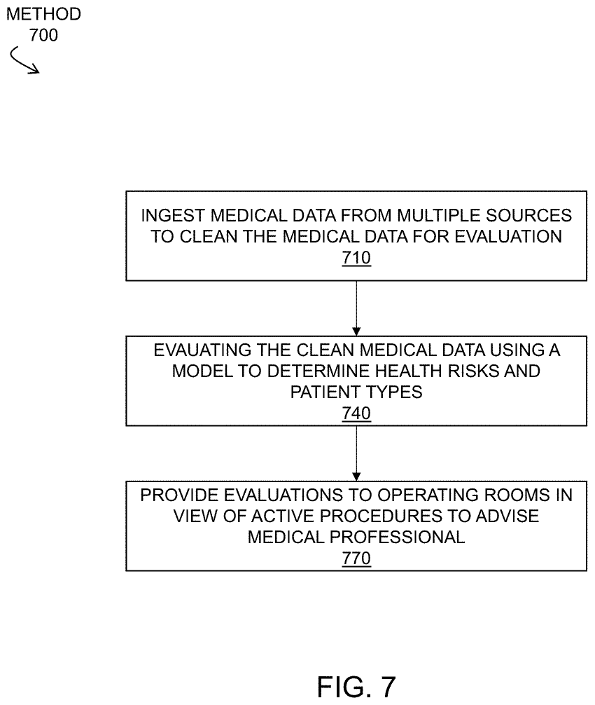

FIG. 7 illustrates a diagram of a method 700 according to one or more embodiments. The method 700 is an example of an operation of the clinical engine 101 described herein. For ease of explanation, reference is made to the items of FIGS. 1-6.

At block 740, the clinical engine 101 ingests medical data (e.g., the clinical information 102) from multiple sources (e.g., the onsite device 105) to clean the medical data for evaluation. The clinical engine 101 receives and associates raw measurement data, such as from a diagnostic device or specific preoperative instrument, with a patient. This can be repeated thousands of times across all patients to create a corpus of clinical information 102.

For instance, the medical data can include precision measurement data regarding pre-lens placement information related to final patient vision comprising the zero or near zero post-operative manifest refraction. The precision measurement data can be derived from optical coherent tomography (OCT), which is a three dimensional imagining technique that uses individual A-scans to identify anatomical surfaces of a human eye. That is, each A-scan acquires surface information for the anterior cornea, posterior cornea, iris, anterior lens, and posterior lens. The OCT performs over 10,000 A-scans to get high resolution data covering the full volume of the anterior segment. The full volume is constructed from the scanned 3-D surfaces. Independent scans are then completed to provide axial and sagittal cross-sections. The OCT scans can be provided on a display to a surgeon (e.g., the medical professional 145). According to one or more embodiments, OCT measurements of the CATALYS™ Precision Laser System can include 3D mappings of the optical surfaces of most of the anterior segment, i.e., front and back curves of the cornea and front back curves of the crystalline lens. The different optical surfaces are registered to each other in x-, y-, and z-dimensions. The registered surfaces are examples of the clinical information 102 exploited by the clinical engine 101.

According to one embodiment and in addition to the scanning operations described herein, printouts from sources (e.g., specific preoperative instruments) can be scanned using optical character recognition (OCR) to extract medical data into a structured database of the clinical engine 101. Further, the clinical engine 101 includes an ability (e.g., using scraping software) to extract online interocular lens calculators to double check medical data from the sources.

At block 740, the clinical engine 101 evaluates the clean medical data using a model (e.g., the model 141) to determine health risks and patient types. The clinical engine 101 trains, such as with respect to the clean medical data of block 710. This training of the clinical engine 101 and the model 141 can also include parsing, analyzing, merging, and correlating of the clean medical data collected. The model 141 can be an unsupervised learning model, such as a self-discover algorithm, or a supervised learning model, such as a support-vector machine (SVM), that analyzes the clean medical data. Further, the model 141 can employ any combination of classification, clustering, regression, anomaly detection, data cleaning, reinforcement learning, structured prediction, feature engineering or learning, semi-supervised learning, decision trees, linear regression, neural or artificial neural networks, logistic regression, recursive selection, relevance vector, and support vector operations, or the like.

At block 770, the clinical engine 101 provide evaluations to operating rooms in view of active procedures to advice medical professional. According to one or more embodiments, the clinical engine 101 can provide optical coherence tomography density evaluation, cataract geometry, corneal anterior and posterior curvatures, etc.

According to one or more embodiments, a system is provided. The system includes a memory configured to store processor executable instructions for a clinical engine; and at least one processor coupled to the memory. The at least one processor is configured to execute the clinical engine to cause the system to register one or more patient profiles, each of the one or more patient profiles comprising a unique identification; aggregate one or more data instances from one or more devices that performed one or more procedures, each of the one or more data instances being associated with a code that includes the unique identification, to produce clinical information; and process the clinical information to determine and associate health or procedure risks with one or more patient types.

According to one or more embodiments and/or any of the system embodiments herein, the clinical engine can aggregate one or more instances of un-coded data relative to one or more second medical procedures.

According to one or more embodiments and/or any of the system embodiments herein, a first instance of the one or more data instances can include coded paperwork or coded data.

According to one or more embodiments and/or any of the system embodiments herein, the clinical engine can process the code of the coded paperwork or the coded data to aggregate the one or more data instance into the clinical information.

According to one or more embodiments and/or any of the system embodiments herein, each unique identification of the one or more patient profiles can include non-identifying demographic information.

According to one or more embodiments and/or any of the system embodiments herein, the clinical engine can provide the health or procedure risks to a clinical device or a user device.

According to one or more embodiments and/or any of the system embodiments herein, the one or more patient types can include at least one of preoperative patients, postoperative patients, intraoperative patients, patients with a known pathology, patients undergoing screening, patients undergoing emergency evaluation, and patients undergoing routine examination.

According to one or more embodiments and/or any of the system embodiments herein, the one or more procedures can include at least one of a medical procedure, a routine procedure, and a surgical procedure.

According to one or more embodiments and/or any of the system embodiments herein, a user device, a clinical device, or a cloud environment can include the at least one or more processors.

According to one or more embodiments and/or any of the system embodiments herein, the clinical engine can execute machine learning or artificial intelligence operations to process the patient data and determine the health or procedure risks.

According to one or more embodiments, a method implemented by a clinical engine system stored as processor executable instructions that are executed by at least one processor is provided. The method includes registering one or more patient profiles, each of the one or more patient profiles comprising a unique identification; aggregating one or more data instances from one or more devices that performed one or more procedures, each of the one or more data instances being associated with a code that includes the unique identification, to produce clinical information; and processing the clinical information to determine and associate health or procedure risks with one or more patient types.

According to one or more embodiments and/or any of the method embodiments herein, the clinical engine can aggregate one or more instances of un-coded data relative to one or more second medical procedures.

According to one or more embodiments and/or any of the method embodiments herein, a first instance of the one or more data instances can include coded paperwork or coded data.

According to one or more embodiments and/or any of the method embodiments herein, the clinical engine can process the code of the coded paperwork or the coded data to aggregate the one or more data instance into the clinical information.

According to one or more embodiments and/or any of the method embodiments herein, each unique identification of the one or more patient profiles can include non-identifying demographic information.

According to one or more embodiments and/or any of the method embodiments herein, the clinical engine can provide the health or procedure risks to a clinical device or a user device.

According to one or more embodiments and/or any of the method embodiments herein, the one or more patient types can include at least one of preoperative patients, postoperative patients, intraoperative patients, patients with a known pathology, patients undergoing screening, patients undergoing emergency evaluation, and patients undergoing routine examination.

According to one or more embodiments and/or any of the method embodiments herein, the one or more procedures can include at least one of a medical procedure, a routine procedure, and a surgical procedure.

According to one or more embodiments and/or any of the method embodiments herein, a user device, a clinical device, or a cloud environment can include the at least one or more processors.

According to one or more embodiments and/or any of the method embodiments herein, the clinical engine can execute machine learning or artificial intelligence operations to process the patient data and determine the health or procedure risks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. A computer readable medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Examples of computer-readable media include electrical signals (transmitted over wired or wireless connections) and computer-readable storage media. Examples of computer-readable storage media include, but are not limited to, a register, cache memory, semiconductor memory devices, magnetic media such as internal hard disks and removable disks, magneto-optical media, optical media such as compact disks (CD) and digital versatile disks (DVDs), a random access memory (RAM), a read-only memory (ROM), an erasable programmable read-only memory (EPROM or Flash memory), a static random access memory (SRAM), and a memory stick. A processor in association with software may be used to implement a radio frequency transceiver for use in a terminal, base station, or any host computer.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one more other features, integers, steps, operations, element components, and/or groups thereof.

The descriptions of the various embodiments herein have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system comprising:
a memory configured to store processor executable instructions for a clinical engine; and
at least one processor coupled to the memory,
wherein the at least one processor is configured to execute the clinical engine to cause the system to:
register one or more patient profiles, each of the one or more patient profiles comprising a unique identification;
aggregate one or more data instances from one or more devices that performed one or more procedures, each of the one or more data instances being associated with a code that includes the unique identification, to produce clinical information; and
process the clinical information to determine and associate health or procedure risks with one or more patient types.

2. The system of claim 1, wherein the clinical engine aggregate one or more instances of un-coded data relative to one or more second medical procedures.

3. The system of claim 1, wherein a first instance of the one or more data instances comprises coded paperwork or coded data.

4. The system of claim 3, wherein the clinical engine processes the code of the coded paperwork or the coded data to aggregate the one or more data instance into the clinical information.

5. The system of claim 1, wherein each unique identification of the one or more patient profiles comprise non-identifying demographic information.

6. The system of claim 1, wherein the clinical engine provides the health or procedure risks to a clinical device or a user device.

7. The system of claim 1, wherein the one or more patient types comprises one or more patient types comprises at least one of preoperative patients, postoperative patients, intraoperative patients, patients with a known pathology, patients undergoing screening, patients undergoing emergency evaluation, and patients undergoing routine examination.

8. The system of claim 1, wherein the one or more procedures comprises at least one of a medical procedure, a routine procedure, and a surgical procedure.

9. The system of claim 1, wherein a user device, a clinical device, or a cloud environment comprises the at least one or more processors.

10. The system of claim 1, wherein the clinical engine executes machine learning or artificial intelligence operations to process the patient data and determine the health or procedure risks.

11. A method implemented by a clinical engine system stored as processor executable instructions that are executed by at least one processor, the method comprising:
registering one or more patient profiles, each of the one or more patient profiles comprising a unique identification;
aggregating one or more data instances from one or more devices that performed one or more procedures, each of the one or more data instances being associated with a code that includes the unique identification, to produce clinical information; and
processing the clinical information to determine and associate health or procedure risks with one or more patient types.

12. The method of claim 11, wherein the clinical engine aggregate one or more instances of un-coded data relative to one or more second medical procedures.

13. The method of claim 11, wherein a first instance of the one or more data instances comprises coded paperwork or coded data.

14. The method of claim 13, wherein the clinical engine processes the code of the coded paperwork or the coded data to aggregate the one or more data instance into the clinical information.

15. The method of claim 11, wherein each unique identification of the one or more patient profiles comprise non-identifying demographic information.

16. The method of claim 11, wherein the clinical engine provides the health or procedure risks to a clinical device or a user device.

17. The method of claim 11, wherein the one or more patient types comprises at least one of preoperative patients, postoperative patients, intraoperative patients, patients with a known pathology, patients undergoing screening, patients undergoing emergency evaluation, and patients undergoing routine examination.

18. The method of claim 11, wherein the one or more procedures comprises at least one of a medical procedure, a routine procedure, and a surgical procedure.

19. The method of claim 11, wherein a user device, a clinical device, or a cloud environment comprises the at least one or more processors.

20. The method of claim 11, wherein the clinical engine executes machine learning or artificial intelligence operations to process the patient data and determine the health or procedure risks.
